(19) United States
(12) Patent Application Publication
(10) Pub. No.: US 2025/0295599 A1
(43) Pub. Date: Sep. 25, 2025

(54) PROBIOTICS ENCAPSULATED WITH CALCIUM CARBONATE AND METHOD FOR PREPARING SAME

(71) Applicant: KOREA MARITIME UNIVERSITY INDUSTRY-ACADEMIC COOPERATION FOUNDATION, Busan (KR)

(72) Inventors: Myoung Jin KIM, Busan (KR); You Jeong LEE, Busan (KR); Se Hun KIM, Seoul (KR); Seon Mi SHIN, Busan (KR)

(73) Assignee: KOREA MARITIME UNIVERSITY INDUSTRY-ACADEMIC COOPERATION FOUNDATION, Busan (KR)

(21) Appl. No.: 18/863,340

(22) PCT Filed: Sep. 21, 2023
(86) PCT No.: PCT/KR2023/014376

§ 371 Date: Nov. 6, 2024

(30) Foreign Application Priority Data
Sep. 30, 2022 (KR) 10-2022-0124896

(51) Int. Cl. A61K 9/50; A61K 9/16; A61K 9/19; A61K 35/745; A61K 35/747

(52) U.S. Cl. CPC A61K 9/501; A61K 9/1611; A61K 9/19; A61K 35/745; A61K 35/747

(57) ABSTRACT

Probiotics encapsulated with calcium carbonate, includes: calcium carbonate; and probiotics. The probiotics encapsulated with calcium carbonate have a particle size of 0.9 to 9.2 μm. The probiotics encapsulated with calcium carbonate have a calcium carbonate content of 17% to 98%. The probiotics encapsulated with calcium carbonate have an intestine-reaching cell count of 8 to 11 Log CFU/g. The calcium carbonate reacts with bile and is converted into hydroxyapatite.

### CROSS-REFERENCE TO PRIOR APPLICATIONS

This Application is a National Stage Patent Application of PCT International Application No. PCT/KR2023/014376 (file on Sep. 21, 2023), which claims priority to Korean Patent Application No. 10-2022-0124896 (filed on Sep. 30, 2022), which are all hereby incorporated by reference in their entirety.

### BACKGROUND

The present invention relates to probiotics encapsulated with calcium carbonate and a method for preparing the same, and more specifically, to probiotics encapsulated with calcium carbonate, which improve the intestinal reach rate, probiotic stability during freeze-drying, and storage stability by encapsulating probiotics with calcium carbonate, and a method for preparing the same.

Probiotics is a general term for live bacteria that are beneficial to the human body when consumed in appropriate amounts, and the term probiotics refers to bacteria that are beneficial to our body. Most probiotics known to date are lactic acid bacteria. Probiotics, such as lactic acid bacteria or beneficial bacteria, survive the gastric acid and bile acid in the body and reach the small intestine, where they proliferate and settle. Thereafter, probiotics exhibit beneficial effects on health in the intestine where they settle, so these probiotics must be non-toxic and non-pathogenic.

What is most important in developing probiotic products is to allow the probiotics to reach the intestines safely while still alive. This is because probiotics themselves are composed of proteins, and when they are administered into the body, the cell membrane is damaged by gastric acid and bile acid, preventing the beneficial functions of the probiotics from being exerted.

Most commercially available probiotics products solve this problem by adding alginate, proteins, polysaccharides, and the like to multi-coat the probiotics. However, when probiotics are protected using the conventional technology, there is a problem that the production cost increases due to multi-coating. Moreover, there are fatal problems that the coating is deformed during the freeze-drying process and the probiotics become dead.

Accordingly, there is a need to develop probiotic products that can effectively reach the intestines while protecting probiotics more easily.

### SUMMARY

An object of the present invention is to provide probiotics encapsulated with calcium carbonate, which improves the intestinal reach rate, probiotic stability during freeze-drying, and storage stability by encapsulating probiotics with calcium carbonate.

Another object of the present invention is to provide probiotic powder prepared by freeze-drying the probiotics encapsulated with the calcium carbonate.

Still another object of the present invention is to provide a method for preparing the probiotics encapsulated with calcium carbonate.

The technical problems to be solved by the present invention are not limited to the above-mentioned technical problems, and other technical problems that are not mentioned may be clearly understood by those skilled in the art from the description of the present invention.

The present invention provides probiotics encapsulated with calcium carbonate, including: calcium carbonate; and probiotics.

In the present invention, the probiotics include one or more microorganisms selected from the group consisting of Lactiplantibacillus plantarum subsp. plantarum (KCTC3108), Lacticaseibacillus casei (KCTC3110), Limosilactobacillus fermentum (KCTC3112), Bifidobacterium longum subsp. longum (KCTC3128), Lactobacillus gasseri (KCTC3144), Lactobacillus acidophilus (KCTC3164), Lactobacillus paragasseri (KCTC3172), Lacticaseibacillus rhamnosus (KCTC3237), Bifidobacterium breve (KCTC3419), Limosilactobacillus reuteri (KCTC3594), Lactobacillus delbrueckii subsp. bulgaricus(KCTC3635), Lactococcus lactis subsp. lactis (KCTC3769), Enterococcus faecalis (KCTC5191), Bifidobacterium animalis subsp. lactis (KCTC5854), Enterococcus faecium (KCTC13225), and Lactobacillus helveticus (KCTC15060).

In the present invention, the probiotics encapsulated with calcium carbonate have a particle size of 0.9 to 9.2 μm.

In the present invention, the probiotics encapsulated with calcium carbonate have a calcium carbonate content of 17% to 98%.

In the present invention, the probiotics encapsulated with calcium carbonate have an intestine-reaching cell count of 8.2 to 10.4 Log CFU/g.

In the present invention, the calcium carbonate reacts with bile and is converted into hydroxyapatite.

In the present invention, the probiotics encapsulated with calcium carbonate have an encapsulation yield of 96% to 100%, and the encapsulation yield is calculated by Equation 1 below:

$$E ( \% ) = [ ( {N}_{0} - {N}_{1} ) \div {N}_{0} ] \times 100 \qquad [ Equation 1 ]$$

(In the calculation formula, E(%) is the encapsulation yield, N₀ is the number of cells used in the probiotics encapsulation process (CFU/g), and N₁ is the number of cells remaining in the solution after encapsulation (CFU/g), that is, the number of cells that have not settled inside capsules (CFU/g).)

In addition, the present invention provides probiotic powder prepared by freeze-drying probiotics encapsulated with calcium carbonate according to the present invention.

In addition, the present invention provides a liquid probiotic preparation prepared by adding probiotics encapsulated with calcium carbonate according to the present invention to a liquid formulation.

In addition, the present invention provides a method for preparing probiotics encapsulated with calcium carbonate, including: culturing probiotics; and mixing a calcium source, a carbonate source, and the cultured probiotics and allowing them to react to produce probiotics encapsulated with calcium carbonate.

In the present invention, the present invention provides the method for preparing probiotics encapsulated with calcium carbonate, further including: freeze-drying and powdering the probiotics encapsulated with calcium carbonate.

In the present invention, the calcium source is one or more calcium sources selected from the group consisting of CaCl₂, Ca(NO₃)₂, CaO, and organic acid calcium.

In the present invention, the carbonate source is one or more carbonate sources selected from the group consisting of Na₂CO₃, K₂CO₃, (NH₄)₂CO₃, and CO₂ (g).

In the present invention, the probiotics include one or more microorganisms selected from the group consisting of Lactiplantibacillus plantarum subsp. plantarum (KCTC3108), Lacticaseibacillus casei (KCTC3110), Limosilactobacillus fermentum (KCTC3112), Bifidobacterium longum subsp. longum (KCTC3128), Lactobacillus gasseri (KCTC3144), Lactobacillus acidophilus (KCTC3164), Lactobacillus paragasseri (KCTC3172), Lacticaseibacillus rhamnosus (KCTC3237), Bifidobacterium breve (KCTC3419), Limosilactobacillus reuteri (KCTC3594), Lactobacillus delbrueckii subsp. bulgaricus (KCTC3635), Lactococcus lactis subsp. lactis (KCTC3769), Enterococcus faecalis (KCTC5191), Bifidobacterium animalis subsp. lactis (KCTC5854), Enterococcus faecium (KCTC13225), and Lactobacillus helveticus (KCTC15060).

In the present invention, the calcium source has a concentration of 50 to 60000 ppm.

In the present invention, the carbonate source has a concentration of 50 to 60000 ppm.

The present invention can provide probiotics encapsulated with calcium carbonate, which improve the intestinal reach rate, probiotic stability during freeze-drying, and storage stability by encapsulating probiotics with calcium carbonate, and a method for preparing the same.

In addition, the present invention can provide probiotic powder prepared by freeze-drying the probiotics encapsulated with calcium carbonate.

In addition, the present invention can provide a probiotic liquid formulation prepared by adding the probiotics encapsulated with calcium carbonate to a liquid formulation.

In addition, the present invention can provide a method for preparing the probiotics encapsulated with calcium carbonate.

The effects of the present invention are not limited to the above-mentioned effects, and other effects that are not mentioned will be clearly understood by those skilled in the art from the description of the claims.

### BRIEF DESCRIPTION OF THE DRAWINGS

FIG. 1 shows a schematic diagram illustrating the process of preparing probiotics encapsulated with calcium carbonate (PEC).

FIG. 2 shows a graph illustrating the X-ray diffraction (XRD) analysis results of PEC according to calcium solution concentration.

FIG. 3 shows a graph illustrating the XRD analysis results of PEC according to the presence or absence of freeze-drying.

FIG. 4 shows a graph illustrating the change in the particle size of PEC according to calcium solution concentration.

FIG. 5 shows a graph illustrating the change in Fourier-transform infrared spectroscopy (FT-IR) peaks of PEC according to calcium solution concentration.

FIG. 6 shows a graph illustrating the thermogravimetric analysis (TGA) analysis results of PEC according to calcium solution concentration.

FIG. 7 shows a graph illustrating the scanning electron microscope-energy dispersive X-ray spectroscopy (SEM-EDS) analysis results of PEC according to calcium solution concentration.

FIG. 8 shows a graph illustrating the confocal laser scanning microscopy (CLSM) analysis results of PEC according to calcium solution concentration.

FIG. 9 shows a graph illustrating the SEM-EDS results of PEC after acid resistance and bile resistance evaluation.

FIG. 10 shows a graph illustrating the FT-IR analysis results of PEC after acid resistance and bile resistance evaluation.

FIG. 11 shows a graph illustrating the XRD analysis results of PEC after acid resistance and bile resistance evaluation.

FIG. 12 shows a graph illustrating the X-ray fluorescence (XRF) analysis results of PEC before and after acid resistance and bile resistance evaluation.

FIG. 13 shows a graph illustrating the results of analyzing the storage stability of PEC according to the calcium solution concentration.

FIG. 14 shows a schematic diagram illustrating the experimental process of preparing a constipation-induced animal model.

FIG. 15 shows a graph illustrating the effect of PEC on the number of feces in a constipation-induced animal model.

FIG. 16 shows a graph illustrating the effect of PEC on fecal weight in a constipation-induced animal model.

FIG. 17 shows a graph illustrating the effect of PEC on fecal moisture content in a constipation-induced animal model.

FIG. 18 shows a graph illustrating the effect of PEC on digestive tract transfer rate in a constipation-induced animal model.

FIG. 19 shows a graph illustrating the change in pH and intestinal reach rate according to the storage period of a liquid formulation containing PEC.

### DETAILED DESCRIPTION

The terms used herein are selected as general terms that are currently widely used as much as possible while considering the functions in the present invention, but they may vary depending on the intention or precedent of those of ordinary skill in the art, the emergence of new technology, or the like. In addition, in certain cases, there are terms arbitrarily selected by the applicant, and in this case, the meaning will be described in the corresponding part of the detailed description of the invention. Therefore, the terms used in the present invention should be defined based on the meaning of the terms and the overall content of the present invention, rather than simply the names of the terms.

Unless otherwise defined, all terms, including technical and scientific terms used herein, have the same meaning as generally understood by one of ordinary skill in the art to which the present invention pertains. Terms, such as those defined in commonly used dictionaries, should be interpreted as having a meaning that is consistent with their meaning in the context of the relevant art and are not interpreted in an idealized or overly formal sense unless clearly so defined in the present invention.

Numerical ranges are inclusive of the values defined therein. Every maximum numerical limitation given throughout the present specification includes every lower numerical limitation, as if such lower numerical limitations were expressly written. Every minimum numerical limitation given throughout the present specification includes every higher numerical limitation, as if such higher numerical limitations were expressly written. Every numerical limitation given throughout the present specification will include every better numerical range within the broader numerical range, as if the narrower numerical limitations were expressly written.

Hereinafter, the present invention will be described in detail.

### Probiotics Encapsulated With Calcium Carbonate (PEC)

The present invention provides probiotics encapsulated with calcium carbonate, including: calcium carbonate; and probiotics. In the present invention, the calcium carbonate may be vaterite or calcite.

The probiotics may be one or more microorganisms selected from the group consisting of Lactiplantibacillus plantarum subsp. plantarum (KCTC3108), Lacticaseibacillus casei (KCTC3110), Limosilactobacillus fermentum (KCTC3112), Bifidobacterium longum subsp. longum (KCTC3128), Lactobacillus gasseri (KCTC3144), Lactobacillus acidophilus (KCTC3164),Lactobacillus paragasseri (KCTC3172), Lacticaseibacillus rhamnosus (KCTC3237), Bifidobacterium breve (KCTC3419), Limosilactobacillus reuteri (KCTC3594), Lactobacillus delbrueckii subsp. bulgaricus (KCTC3635), Lactococcus lactis subsp. lactis (KCTC3769), Enterococcus faecalis (KCTC5191), Bifidobacterium animalis subsp. lactis (KCTC5854), Enterococcus faecium (KCTC13225), and Lactobacillus helveticus (KCTC15060), preferably one or more microorganisms selected from the group consisting of Lactobacillus acidophilus (KCTC3164), Lactiplantibacillus plantarum subsp. plantarum (KCTC3108), Lacticaseibacillus rhamnosus (KCTC 3237), Bifidobacterium breve (KCTC 3419), and Lactococcus lactis subsp. lactis (KCTC 3769), more preferably, a microbial consortium including all of Lactobacillus acidophilus (KCTC3164), Lactiplantibacillus plantarum subsp. plantarum (KCTC3108), Lacticaseibacillus rhamnosus (KCTC 3237), Bifidobacterium breve (KCTC 3419), and Lactococcus lactis subsp. lactis (KCTC 3769).

The probiotics encapsulated with calcium carbonate of the present invention may be named as probiotics encapsulated with calcium carbonate (PEC), and the particle size of the PEC may be 0.9 to 9.2 μm, preferably 1.5 to 9.2 μm, and more preferably 7.0 to 9.0 μm.

The PEC may have a calcium carbonate content of 17 to 98%, and preferably 75 to 98%. In addition, the weight ratio of the probiotics and calcium carbonate of the PEC may be 1:0.2 to 17.6, and preferably 1:3 to 17.6.

The PEC may show a stretching vibration peak of OH at 3280 cm⁻¹, which is a characteristic peak of probiotics, when analyzed by Fourier transform infrared spectroscopy (FT-IR), and peaks of CH, C═C, and CO at 2927, 1636, and 1036 cm⁻¹, respectively. In addition, the PEC may show peaks at 1386, 872, and 712 cm⁻¹, which are characteristic peaks of calcium carbonate, when analyzed by FT-IR.

The encapsulation yield of the PEC may be calculated according to the following Equation 1, and the PEC of the present invention may have an encapsulation yield of 96% to 100%.

$$E ( \% ) = [ ( {N}_{0} - {N}_{1} ) \div {N}_{0} ] \times 100 \qquad [ Equation 1 ]$$

(In the calculation formula, E(%) is the encapsulation yield, N₀ is the number of cells used in the probiotics encapsulation process (CFU/g), and N₁ is the number of cells remaining in the solution after encapsulation (CFU/g), that is, the number of cells that have not settled inside capsules (CFU/g).)

The probiotic content (Log CFU/g) of the PEC may be 8 to 11.

The intestine-reaching cell count of the PEC may be calculated according to the following Equation 2, and the PEC of the present invention may have different intestinal reach rates depending on the concentration of the calcium solution used during preparation. More specifically, the PEC prepared using 50 to 200 ppm and 60,000 ppm calcium solutions may have an intestine-reaching cell count of 8 to 9.5 Log CFU/g, and the PEC prepared using 400 to 40,000 ppm calcium solutions may have an intestine-reaching cell count of 10 to 11 Log CFU/g.

$$I ( \% ) = [ {N}_{2} \div ( {N}_{0} - {N}_{1} ) ] \times 100 \qquad [ Equation 2 ]$$

(In Equation 2, N₂ represents the finally released cell count (CFU/g) after consecutive tests with simulated gastric fluid (SGF) and simulated intestinal fluid (SIF), and N₀-N₁ represents the cell count (CFU/g) encapsulated with calcium carbonate.)

In the PEC of the present invention, the calcium carbonate included in the PEC may react with bile and be converted into hydroxyapatite (Ca₁₀(PO₄)₆(OH)₂).

In the PEC of the present invention, the calcium carbonate included in the PEC may serve as not only an encapsulating agent but also a freeze-drying protection agent. In other words, probiotics encapsulated with calcium carbonate may have a higher survival rate of probiotics during freeze-drying compared to non-encapsulated probiotics.

The present invention may provide probiotic powder prepared by freeze-drying the above-described PEC.

In addition, the present invention may provide a probiotic liquid formulation prepared by adding the above-described PEC to a liquid formulation, and the liquid formulation may be milk or milk with a pH adjusted to 7.5.

### Method for Preparing PEC

The present invention provides a method for preparing PEC, including: culturing probiotics; and mixing a calcium source, a carbonate source, and the cultured probiotics and allowing them to react to produce PEC. In addition, the method for preparing PEC may further include freeze-drying and powdering the PEC.

The culturing probiotics is a step of culturing a probiotics to be used in PEC, and the probiotics may be one or more microorganisms selected from the group consisting of Lactiplantibacillus plantarum subsp. plantarum (KCTC3108), Lacticaseibacillus casei (KCTC3110), Limosilactobacillus fermentum (KCTC3112), Bifidobacterium longum subsp. longum (KCTC3128), Lactobacillus gasseri (KCTC3144), Lactobacillus acidophilus (KCTC3164), Lactobacillus paragasseri (KCTC3172 ), Lacticaseibacillus rhamnosus (KCTC3237), Bifidobacterium breve (KCTC3419), Limosilactobacillus reuteri (KCTC3594), Lactobacillus delbrueckii subsp. bulgaricus (KCTC3635), Lactococcus lactis subsp. lactis (KCTC3769), Enterococcus faecalis (KCTC5191), Bifidobacterium animalis subsp. lactis (KCTC5854), Enterococcus faecium (KCTC13225), and Lactobacillus helveticus (KCTC15060), preferably one or more microorganisms selected from the group consisting of Lactobacillus acidophilus (KCTC3164), Lactiplantibacillus plantarum subsp. plantarum (KCTC3108), Lacticaseibacillus rhamnosus (KCTC 3237), Bifidobacterium breve (KCTC 3419), and Lactococcus lactis subsp. lactis (KCTC 3769), more preferably, a microbial consortium including all of Lactobacillus acidophilus (KCTC3164), Lactiplantibacillus plantarum subsp. plantarum (KCTC3108), Lacticaseibacillus rhamnosus (KCTC 3237), Bifidobacterium breve (KCTC 3419), and Lactococcus lactis subsp. lactis (KCTC 3769).

The probiotics may be used by inoculating them into an autoclaved De Man, Rogosa, and Sharpe (MRS) broth medium and culturing them at 35 to 39° C. and 100 to 300 rpm for 48 to 72 hours, and then subculturing them.

The mixing a calcium source, a carbonate source, and the cultured probiotics and allowing them to react to produce PEC may be a step of mixing and stirring the calcium source, the carbonate source, and the probiotics to produce PEC.

The calcium source may be a calcium solution, and more specifically, may include one or more substance selected from the group consisting of CaCl₂, Ca(NO₃)₂, CaO, and an organic acid calcium. Preferably, the calcium source may be CaCl₂. The concentration of the calcium source may be 50 to 60,000 ppm, and preferably 400 to 40,000 ppm.

The carbonate source may be a carbonate solution, and more specifically, may include one or more substances selected from the group consisting of Na₂CO₃, K₂CO₃, (NH₄)₂CO₃, and CO₂ (g). Preferably, the carbonate source may be Na₂CO₃. The concentration of the carbonate source may be 50 to 60,000 ppm, preferably 400 to 40,000 ppm.

The freeze-drying and powdering the PEC may be a step of producing probiotic powder by freeze-drying the PEC. The freeze-drying may mean filtering the PEC, rapidly freezing it at −70 to −50° C. for one hour, and then freeze-drying it at −90 to −60° C. for 20 to 28 hours.

Hereinafter, examples of the present invention will be described in detail, but it is obvious that the present invention is not limited to the following examples.

### Example 1. Preparation of PEC

### 1-1. Strain Selection

Example 1 was implemented to prepare PEC of the present invention. A total of five strains were used to prepare the PEC, and more specifically, Lactobacillus acidophilus (KCTC3164), Lactiplantibacillus plantarum subsp. plantarum (KCTC3108), Lacticaseibacillus rhamnosus (KCTC 3237), Bifidobacterium breve (KCTC 3419), and Lactococcus lactis subsp. lactis (KCTC 3769) were mixed at an equal ratio and used. The five strains were provided by the Korean Collection for Type Cultures (KCTC).

### 1-2. Probiotics Culture

Example 1-2 was implemented to culture the five probiotic strains selected in Example 1-1. The five probiotic strains were cultured in a liquid phase in a probiotics culture medium, and the culture medium used was Difo™ Lactobacilli MRS broth (Becton, Dickinson and Company, France), Difo™ Lactobacilli MRS Agar (Becton, Dickinson and Company, France), and BL Agar (KisanBio, Seoul, Korea). Each strain was prepared as a culture solution of 10¹⁰ CFU/mL or more.

Next, 55 g of MRS broth was added to 1 L of distilled water and sterilized under high pressure at 121° C. for 15 minutes, and then the strain was inoculated. The inoculated medium was cultured in an incubator at 37° C. and 200 rpm for 48 to 72 hours, and then subcultured every week for use. The culture solution was centrifuged at 15,000 rpm, 5° C., and 15 minutes in a centrifuge. After centrifugation, the supernatant was discarded, and the pellet was washed three times with sterilized distilled water and used in the experiment. In addition, 70 g and 60.23 g of MRS agar and BL agar, which are media for measuring the number of viable probiotics, were respectively added to 1 L of distilled water, sterilized in an autoclave at 121° C. for 15 minutes, and then stored at 4° C. for use.

### 1-3. PEC Preparation

PEC was prepared by mixing the five strains cultured in the Example 1-2 with a calcium solution and a carbonate solution. The number of mixed strains (probiotics) was 10.16 Log CFU/g. The PEC was prepared by stirring the mixture of the CaCl₂, Na₂CO₃and the cultured strains at 800 rpm using a magnetic bar.

### 1-4. Preparation of PEC Powder

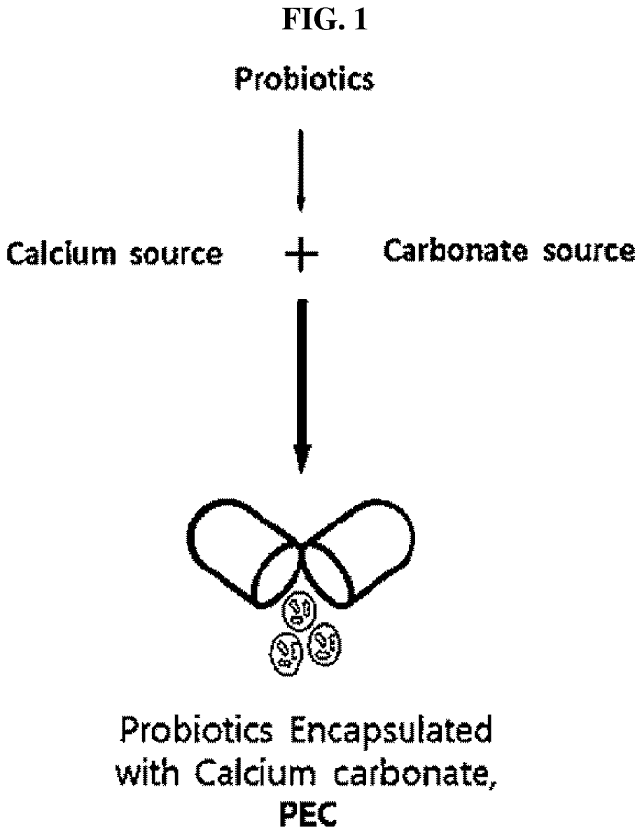

Example 1-4 was implemented to prepare powder of the PEC prepared in the Example 1-3 by freeze-drying. The PEC solution prepared in the Example 1-3 was filtered through a 0.45 μm membrane filter (MCE04547A, HYUNDAI Micro Co.), and then rapidly frozen in a −60° C. freezer for four hours. Thereafter, it was dried for 24 hours under the conditions of 5 mbar and −80° C. using a freeze-dryer (Operon, OPR-FDU-8606, Korea). PEC powder was prepared through the above-described freeze-drying process. The preparation process of PEC disclosed in the Examples 1-1 to 1-4 is schematically illustrated in FIG. 1.

### Experimental Example 1. XRD Analysis of PEC

### 1-1. Changes in Calcium Carbonate Form According to Calcium Solution Concentration

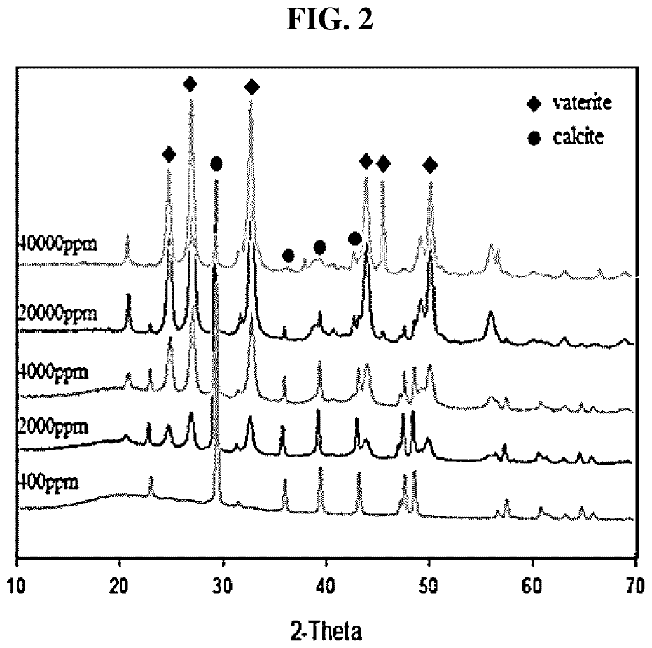

To investigate the changes in the calcium carbonate form of the PEC prepared by varying the calcium solution concentration, Experimental Example 1-1 was implemented. The PEC was prepared in the same manner as in Example 1, except that the calcium solution was prepared by varying the concentration to 400, 2000, 4000, 20000, or 40000 ppm in Experimental Example 1-1. The XRD peaks of the PEC prepared by varying the calcium solution concentration were analyzed using an X-ray diffractometer (XRD, Smart lab, Rigaku). The XRD analysis results are shown as a graph in FIG. 2.

As a result of Experimental Example 1-1, it was confirmed that the material encapsulating the probiotics in the PEC was calcium carbonate in the form of calcite and vaterite crystals. Referring to FIG. 2, the characteristic peak of calcite (indicated by ●) and the characteristic peak of vaterite (indicated by ♦) were confirmed together, and as the calcium solution concentration decreased, the intensity of the characteristic peak of calcite increased and the intensity of the characteristic peak of vaterite decreased.

### 1-2. Changes in Calcium Carbonate Form Depending on Freeze-Drying

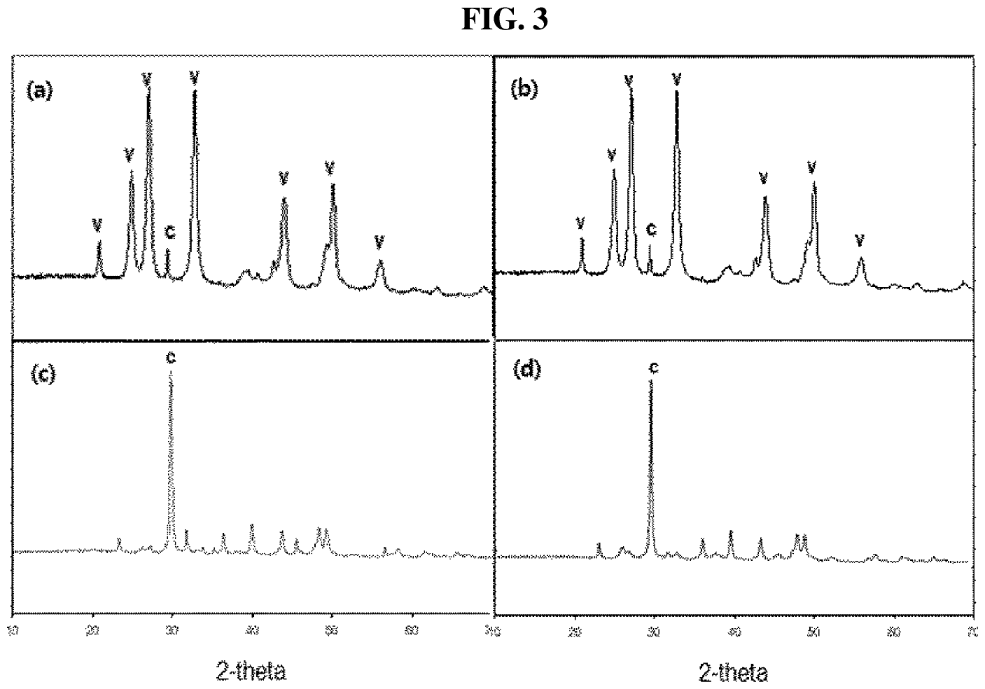

To investigate the changes in the XRD peak of calcium carbonate depending on freeze-drying, Experimental Example 1-2 was implemented. In Experimental Example 1-2, an experiment was performed separately for the PEC with calcite as the calcium carbonate encapsulating probiotics and the PEC with vaterite as the calcium carbonate encapsulating probiotics, and the XRD peaks of the calcium carbonate of the two types of PEC were compared depending on freeze-drying. The freeze-drying was performed in the same manner as in Example 1-4, and the XRD analysis was performed in the same manner as Experimental Example 1-1. The results of the Experimental Example 1-2 are shown as a graph in FIG. 3, and more specifically, FIG. 3A shows the XRD of the sample of probiotics encapsulated with vaterite-type calcium carbonate that was not freeze-dried, FIG. 3B shows the XRD of the sample of probiotics encapsulated with vaterite-type calcium carbonate that was freeze-dried, FIG. 3C shows the XRD of the sample of probiotics encapsulated with calcite-type calcium carbonate that was not freeze-dried, and FIG. 3D shows the XRD of the sample of probiotics encapsulated with calcite-type calcium carbonate that was freeze-dried.

As a result of Experimental Example 1-2, it was confirmed that the XRD peak of PEC did not change depending on freeze-drying. In other words, no change in the form and content of PEC was observed before and after freeze-drying.

### Experimental Example 2. Particle Size Analysis of PEC

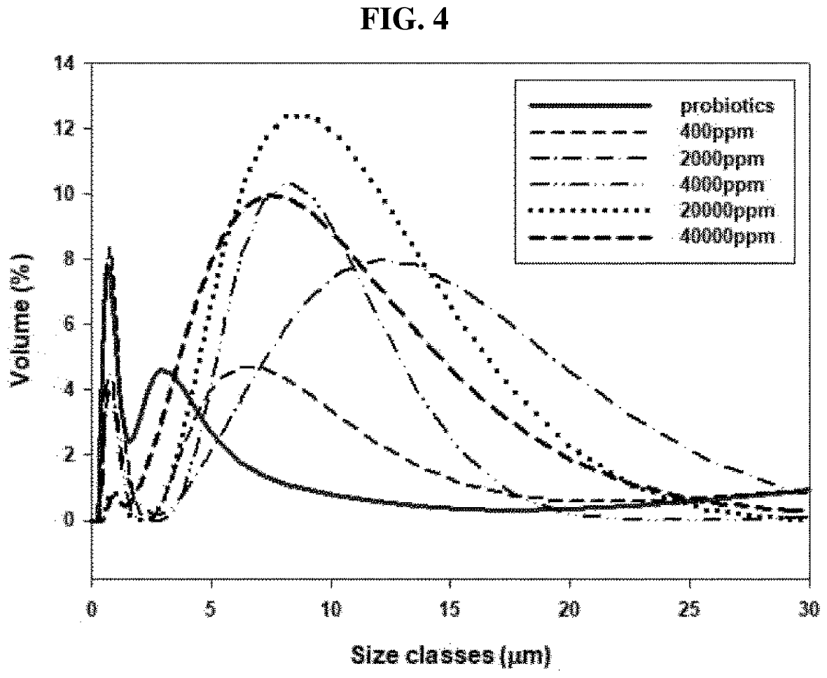

To analyze the particle size of PEC prepared by varying the calcium solution concentration, Experimental Example 2 was implemented. PEC was prepared in the same manner as in Example 1, except that the calcium solution was prepared by varying the concentration to 50, 100, 200, 400, 2000, 4000, 20000, 40000, or 60000 ppm in Experimental Example 2. The particle size of PEC prepared by varying the calcium solution concentration was measured using a particle size analyzer (PSA, Mastersizer 3000, Malvern, UK). The results of Experimental Example 2 are shown in a graph in FIG. 4.

As a result of Experimental Example 2, it was found that, although there was a difference in the particle size depending on the calcium solution concentration, the particle size (D₅₀) of the PEC was 0.9 to 9.2 μm. The particle size distribution according to the calcium solution concentration is shown in Table 1 below.

TABLE 1

| | Particle size (μm) | | |
|---|---|---|---|
| Ca conc. | D10 | D50 | D90 |
| 50 ppm | 0.53 | 0.90 | 4.6 |
| 100 ppm | 0.54 | 0.97 | 6.7 |
| 200 ppm | 0.55 | 1.0 | 7.2 |
| 400 ppm | 0.55 | 1.5 | 35.6 |
| 2000 ppm | 0.67 | 9.2 | 23.8 |
| 4000 ppm | 0.72 | 7.8 | 11.6 |
| 20000 ppm | 4.3 | 8.4 | 15.2 |
| 40000 ppm | 3.0 | 7.2 | 15.3 |
| 60000 ppm | 3.3 | 7.0 | 13.6 |

### Experimental Example 3. FT-IR Analysis of PEC

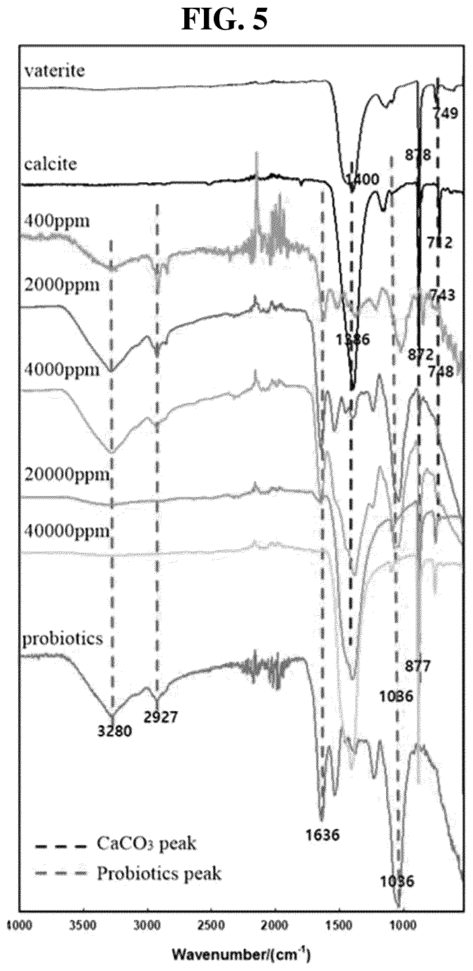

To analyze the FT-IR of PEC, Experimental Example 3 was implemented. PEC was prepared in the same manner as in Example 1, except that the calcium solution was prepared by varying the concentration to 400, 2000, 4000, 20000, or 40000 ppm in Experimental Example 3. The FT-IR of the PEC prepared using the calcium solution at different concentrations was analyzed using a Fourier-transform infrared spectrometer (FT-IR, iS50, Thermo Fisher Scientific, USA), and was analyzed in the spectrum range of 500 to 4000 cm−1. In addition, the FT-IR of calcite, vaterite, and probiotics were also analyzed as comparative groups in Experimental Example 3. The FT-IR analysis results are shown as a graph in FIG. 5.

As a result of Experimental Example 3, the stretching vibration peak of OH appeared at 3280 cm⁻¹ in the spectrum of the PEC, and the CH, C═C, and CO peaks were observed at 2927, 1636, and 1036 cm⁻¹, respectively. All of the peaks are characteristics that appear in probiotics, and as the concentration of the calcium solution increased, the intensity of the corresponding peak decreased, and when the calcium concentration was 20,000 ppm or higher, almost no peak was observed.

In addition, as the calcium solution concentration increased, the intensity of the peaks at 1386, 872, and 712 cm⁻¹ in the spectrum tended to increase, and all of the peaks are characteristics that appear in calcium carbonate. In particular, when the calcium concentration was 20,000 ppm or higher, the intensity of the corresponding peaks of PEC was similar to that of the calcium carbonate of the comparative group. Therefore, it was confirmed through the FT-IR results that the PEC contained both calcium carbonate and probiotics.

### Experimental Example 4. TGA Analysis of PEC

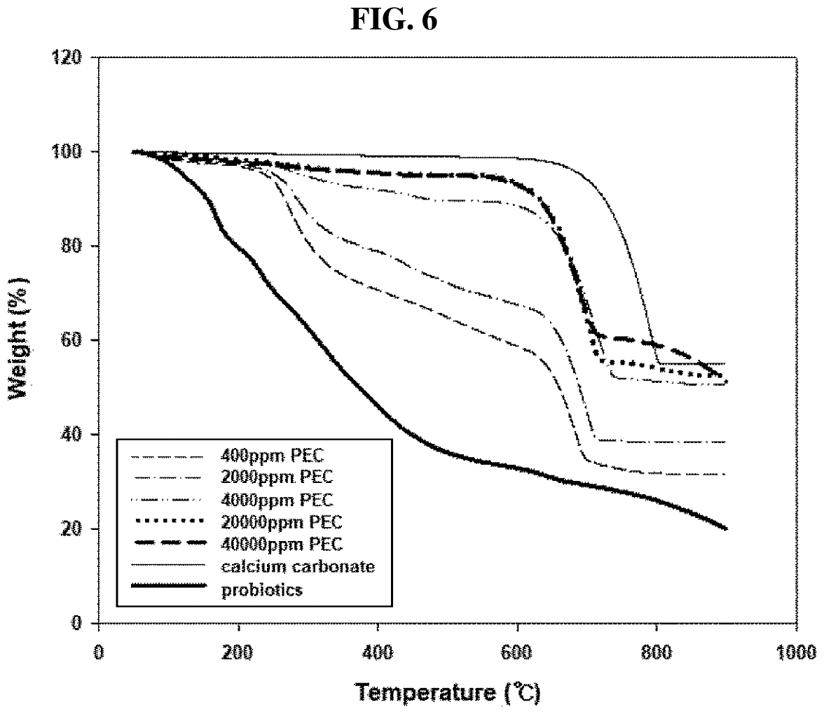

To find out the content ratio of calcium carbonate and probiotics included in PEC, a thermogravimetric analysis (TGA) was performed in Experimental Example 4. PEC was prepared in the same manner as in Example 1, except that the calcium solution was prepared by varying the concentration to 50, 100, 200, 400, 2000, 4000, 20000, 40000, or 60000 ppm in Experimental Example 4. The TGA was measured using a thermogravimetric analyzer (TGA 7, Perkin Elmer, USA), and the weight loss within the range of 50 to 900° C. was measured at a heating rate of 20° C./min. It is known that the boiling point of organic matter is 250 to 500° C., and calcium carbonate is combusted in the range of 500 to 820° C. Therefore, the PEC prepared with a solution having a lower calcium concentration exhibits a greater weight loss at 250 to 500° C. Based on this principle, the calcium carbonate content of the PEC was calculated using the measured values according to Equation 3 below, and the results of Experimental Example 4 are shown as a graph in FIG. 6.

$$calcium carbonate purity ( \% ) = {W}_{CO 2} \times \frac{M {W}_{CaCO 3}}{M {W}_{CO 2}} \qquad [ Equation 3 ]$$

(In Equation 3, W_{CO2} indicates the weight loss (%) of CO2, MW_{CaCO3} indicates the molar mass of CaCO₃ (g/mol), and MW_{CO2} indicates the molar mass of CO₂ (g/mol).)

As a result of Experimental Example 4, it was confirmed that the calcium carbonate content of the PEC was 17% to 98%, and more specifically, the weight ratio of probiotics to calcium carbonate was 1:0.2 to 1:17.6. The calcium carbonate content and the ratio of probiotics to calcium carbonate according to the calcium solution concentration of the PEC are shown in Table 2 below.

TABLE 2

| | Calcium carbonate | Ratio of probiotics to |
|---|---|---|
| Ca conc. | content (%) | calcium carbonate |
| 50 ppm | 38.9 | 1:0.64 |
| 100 ppm | 19.4 | 1:0.24 |
| 200 ppm | 16.8 | 1:0.20 |
| 400 ppm | 75.4 | 1:4.1 |
| 2000 ppm | 81.0 | 1:2.8 |
| 4000 ppm | 92.3 | 1:7.2 |
| 20000 ppm | 98.2 | 1:17.6 |
| 40000 ppm | 86.8 | 1:5.2 |
| 60000 ppm | 91.4 | 1:10.7 |

### Experimental Example 5: Scanning Electron Microscope-Energy Dispersive X-Ray Spectroscopy (SEM-EDS) and X-Ray Fluorescence (XRF) of PEC

### 5-1. SEM-EDS Mapping

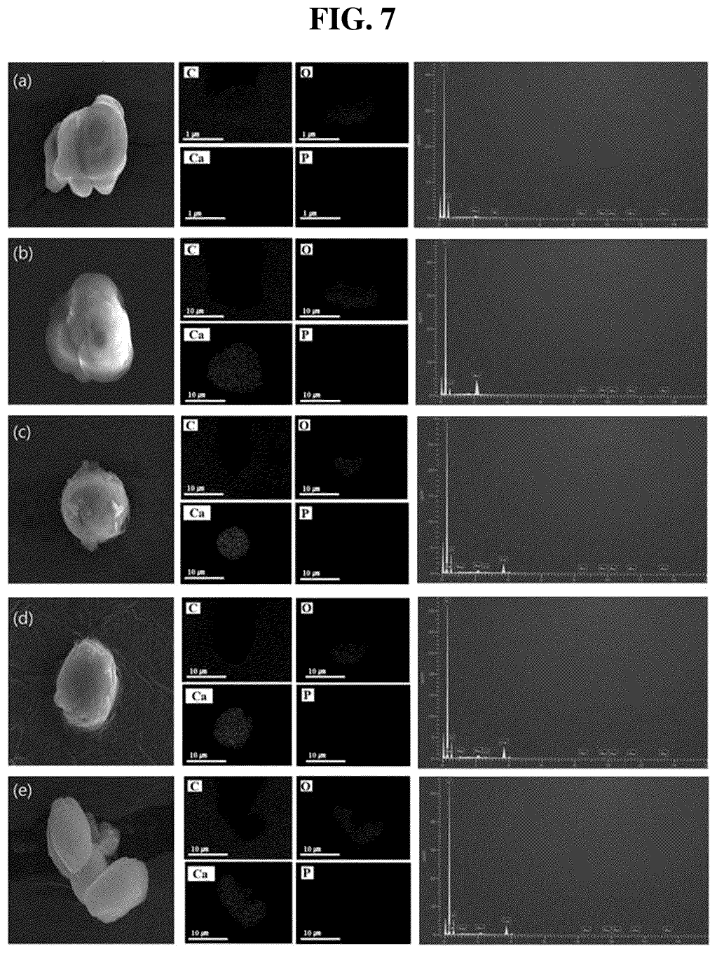

To confirm the surface component and shape of PEC prepared by varying the concentration of calcium solution, Experimental Example 5-1 was implemented. PEC was prepared in the same manner as in Example 1, except that the calcium solution was prepared by varying the concentration to 400, 2000, 4000, 20000, or 40000 ppm in Experimental Example 5-1. The surface component and shape of PEC prepared by varying the calcium solution concentration were analyzed using a scanning electron microscope (SUPRA-40VP, FE-SEM/EDS, ZEISS) and energy dispersive spectroscopy (EDS) mapping. The results of the Experimental Example 5-1 are shown as a graph in FIG. 7, and more specifically, FIGS. 7A, 7B, 7C, 7D, and 7E describe the SEM-EDS results of the PECs prepared by using calcium solutions at concentrations of 400, 2000, 4000, 20000, and 40000 ppm, respectively.

As a result of Experimental Example 5-1, the mainly detected elements, Ca, C, and O, were detected in both calcium carbonate and PEC. In addition, in the case of the PECs prepared in the solutions with the calcium concentrations of 2000 to 40000 ppm, it was confirmed that a large amount of Ca was uniformly dispersed on the surface, but in the case of the PECs prepared in the solution with the calcium concentrations of 400 ppm, it was confirmed that there was almost no Ca on the surface. The reason why the amount of Ca on the surface of the PEC was small is because a relatively large amount of organic matter was present on the surface of the PEC, so that C and O appeared to be much.

### 5-2. XRF Analysis

To identify the components of PEC prepared by varying the concentration of calcium solution, Experimental Example 5-2 was implemented. PEC was prepared in the same manner as Example 1, except that the calcium solution was prepared by varying the concentration to 400, 2000, 4000, 20000, or 40000 ppm in Experimental Example 5-2. The prepared PEC was analyzed using XRF spectrometer (SHIMADZU, XRF-1800). The results of Experimental Example 5-2 are shown in Table 3 below.

TABLE 3

| | Composition (wt %) | | | | | | |
|---|---|---|---|---|---|---|---|
| Element | Probiotics | CaCO₃ | 400 ppm | 2000 ppm | 4000 ppm | 20000 ppm | 40000 ppm |
| Ca | | 98.1724 | 57.2718 | 94.9539 | 97.8204 | 98.7613 | 98.6193 |
| Cl | | 0.8028 | 4.7173 | 0.7728 | | | 0.2113 |
| S | 10.0208 | 0.5788 | 1.3773 | 0.1693 | 0.0979 | 0.0380 | 0.0357 |
| P | 41.0795 | 0.1357 | 13.4568 | 1.4080 | 0.6924 | 0.0596 | |
| Sr | | 0.1789 | | | | | |
| Al | | 0.0703 | | | | | |
| K | 37.2937 | | 13.1873 | 0.2439 | 0.1847 | | |
| Si | 0.3741 | 0.0551 | | | | | |
| Na | 7.0477 | | 6.0123 | 1.4665 | 1.2046 | 1.1410 | 1.1337 |
| Mn | 2.5387 | | 3.9772 | 0.9857 | | | |

As a result of Experimental Example 5-2, the presence of P, K, Na, and Mn, which are components of probiotics included in the PEC, were not detected in the SEM-EDS result of Experimental Example 5-1, but were detected in the XRF result. This supports that the probiotics were included inside the PEC. In particular, K, Na, and Mn were not detected in the XRF result of calcium carbonate, but were detected in the PEC, which can serve as clear evidence that probiotics were encapsulated inside calcium carbonate.

### Experimental Example 6. Confocal Laser Scanning Microscopy (CLSM) Analysis of PEC

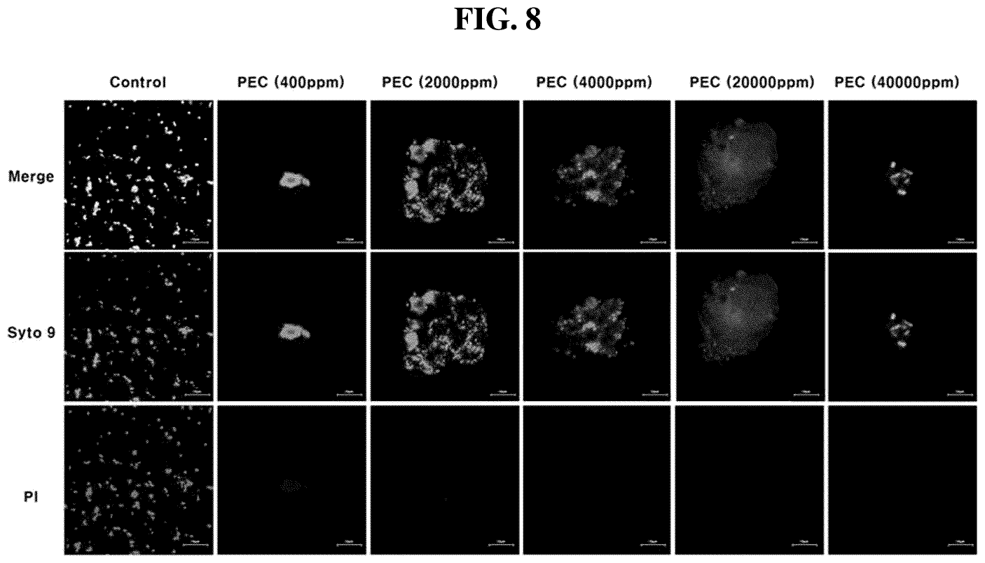

To confirm the survival of probiotics encapsulated inside PEC, a CLSM analysis was performed in Experimental Example 6. PEC was prepared in the same manner as in Example 1, except that calcium solution was prepared by varying the concentration to 400, 2000, 4000, 20000, or 40000 ppm. The survival of the bacteria inside the PEC prepared at each calcium solution concentration was analyzed using confocal laser scanning microscope (CLSM, LSM 800, ZEISS), and the survival of the bacteria was analyzed using a LIVE/DEAD BacLight Bacterial Viability Kit (Thermo Fisher Scientific, MA, USA). The LIVE/DEAD Kit consists of Syto9 and propidium iodide (PI). The Syto9 is membrane-permeable, so it passes through the cell membrane and stains nucleic acids to exhibit green fluorescence, but the PI is membrane-impermeable, so it stains nucleic acids of bacteria with damaged cell membranes and exhibit red fluorescence. Therefore, when Syto9 and PI are mixed and used for staining, live bacteria are stained green by Syto9, and dead bacteria are stained red by PI. In addition, in Experimental Example 6, the probiotics that were not encapsulated with the calcium carbonate and were freeze-dried were used as a comparative group. The results of the Experimental Example 6 are shown as a graph in FIG. 8.

As a result of Experimental Example 6, it was confirmed that the probiotics were encapsulated inside the PEC regardless of the concentration of the calcium solution, and overall, red (PI), which indicates dead bacteria, was not visible, and green (Syto9), which indicates live bacteria, was mostly observed. This means that most of the encapsulated probiotics did not die but survived.

On the other hand, the probiotics that were freeze-dried without encapsulating with calcium carbonate, which was set as a control, showed a live cell: dead cell ratio of approximately 1:1. Therefore, it was shown that in the PEC of the present invention, calcium carbonate served as not only a probiotic encapsulating agent but also as a freeze-drying protecting agent.

### Experimental Example 7. Analysis of Encapsulation Yield of PEC

To confirm the encapsulation yield of PEC prepared by varying the concentration of calcium solution, Experimental Example 7 was implemented. PEC was prepared in the same manner as Example 1, except that the calcium solution was prepared by varying the concentration to 50, 100, 200, 400, 2000, 4000, 10000, 20000, 40000, or 60000 ppm in Experimental Example 7. The encapsulation yield of the prepared PEC was calculated twice per sample by a culture medium method. More specifically, the prepared PEC solution was diluted by mixing it with sterilized distilled water at a ratio of 1:9 (g:mL), and then spread on an agar medium and cultured at 37° C. for 48 hours. Thereafter, the number of viable cells was measured, and the encapsulation yield was calculated according to the following Equation 1. The results of Experimental Example 7 are shown in Table 4 below.

$$E ( \% ) = [ ( {N}_{0} - {N}_{1} ) \div {N}_{0} ] \times 100 \qquad [ Equation 1 ]$$

(In the calculation formula, E(%) is the encapsulation yield, N₀ is the number of cells used in the probiotics encapsulation process (CFU/g), and N₁ is the number of cells remaining in the solution after encapsulation (CFU/g), that is, the number of cells that have not settled inside capsules (CFU/g).)

As a result of Experimental Example 7, although there was a slight difference depending on the concentration of the calcium solution used, an encapsulation yield of more than 97% was obtained from all PECs. In addition, the probiotics content (Log CFU/g) of all PECs was 10.14 to 10.16.

TABLE 4

| | | | | Encapsulation |
|---|---|---|---|---|
| Ca conc | N₀ | N₁ | Encapsulation | yield |
| (ppm) | (Log CFU/g) | (Log CFU/g) | (Log CFU/g) | (%) |
| 50 | 10.16 ± 0.02 | 8.05 ± 0.01 | 10.16 ± 0.02 | 99.50 |
| 100 | 10.16 ± 0.02 | 7.89 ± 0.02 | 10.16 ± 0.05 | 99.74 |
| 200 | 10.16 ± 0.02 | 7.87 ± 0.04 | 10.16 ± 0.03 | 99.76 |
| 400 | 10.16 ± 0.07 | 7.90 ± 0.03 | 10.16 ± 0.03 | 99.44 |
| 2000 | 10.16 ± 0.02 | 8.33 ± 0.05 | 10.15 ± 0.04 | 98.50 |
| 4000 | 10.16 ± 0.05 | 8.65 ± 0.01 | 10.14 ± 0.01 | 96.89 |
| 10000 | 10.16 ± 0.03 | 7.86 ± 0.05 | 10.16 ± 0.03 | 99.95 |
| 20000 | 10.16 ± 0.05 | 8.42 ± 0.03 | 10.15 ± 0.07 | 98.19 |
| 40000 | 10.16 ± 0.10 | 7.86 ± 0.02 | 10.16 ± 0.18 | 99.95 |
| 60000 | 10.16 ± 0.02 | 8.02 ± 0.01 | 10.16 ± 0.01 | 99.54 |

### Experimental Example 8. Analysis of Intestinal Reach Rate of PEC

To confirm the intestinal reach rate of PEC prepared by varying the concentration of calcium solution, Experimental Example 8 was implemented. PEC was prepared in the same manner as in Example 1, except that PEC was prepared by using calcium solution at concentrations of 50, 100, 200, 400, 2000, 4000, 10000, 20000, 40000, or 60000 ppm in Experimental Example 8.

To measure the number of viable bacterial cells released from the prepared PEC through the stomach into the intestine, acid tolerance and bile tolerance tests were conducted consecutively using SGF and SIF. The SGF was prepared based on adjusting the pH value of 0.2% (w/v) NaCl solution to 2 and adding 1 N HCl to simulate the fasting conditions in the stomach. Then, 3.2 g of pepsin was added to prepare the SGF. The SIF was prepared by adjusting the pH value of 0.05 M KH₂PO₄ solution to 7.2 using 1 M NaOH.

After adding the PEC to the SGF, the reaction was performed at 200 rpm for two hours in a shaking incubator at 37° C., and then the solid filtered through a 0.45 μm membrane (MCE04547A, HYUNDAI Micro Co.) was added to the SIF. The SIF test was also performed under the same conditions as the SGF test, and the solution that underwent the two consecutive tests was diluted and spread on an agar medium, and then cultured at 37° C. for 48 hours to measure the number of viable cells. Since the intestinal reach rate is related to the amount of probiotics released by the PEC into the intestines through gastric juice and bile, it was calculated according to Equation 2 below. The results of Experimental Example 8 are shown in Table 5 below.

$$I ( \% ) = [ {N}_{2} \div ( {N}_{0} - {N}_{1} ) ] \times 100 \qquad [ Equation 2 ]$$

(In Equation 2, N₂ represents the finally released cell count (CFU/g) after consecutive tests with SGF and SIF, and N₀-N₁ represents the cell count (CFU/g) encapsulated with calcium carbonate.)

As a result of Experimental Example 8, it was confirmed that the intestinal reach rate of the PEC prepared using a calcium solution at concentrations of 400 to 40,000 ppm was 10.17 to 10.43 based on Log CFU/g, and more than 10 billion CFU of probiotics were released in a live state into the intestine.

It was confirmed that in the PECs with a calcium concentration of 400 to 40,000 ppm, more than 100% of the probiotics injection amount and more than 10 billion CFU/g of probiotics in terms of the cell count were released in a live state into the intestine. Considering that the recommended daily probiotic intake range according to the Ministry of Food and Drug Safety is 8 to 10 based on Log CFU/g, this result, in which the intestinal reach amount, not the intake amount, was 8.17 to 10.43 based on Log CFU/g, indicates that the performance of the PEC developed in the present study is very good.

TABLE 5

| Ca concentration | N₀ | Intestinal reach | Intestinal |
|---|---|---|---|
| (mg/L) | (Log CFU/g) | (Log CFU/g) | reach rate (%) |
| 50 | 10.16 ± 0.02 | 8.18 ± 0.01 | 1.0 |
| 100 | 10.16 ± 0.02 | 9.42 ± 0.31 | 18.2 |
| 200 | 10.16 ± 0.02 | 9.08 ± 0.23 | 8.3 |
| 400 | 10.16 ± 0.07 | 10.17 ± 0.01 | 100 |
| 2000 | 10.16 ± 0.02 | 10.19 ± 0.04 | 100 |
| 4000 | 10.16 ± 0.05 | 10.37 ± 0.03 | 100 |
| 10000 | 10.16 ± 0.05 | 10.43 ± 0.11 | 100 |
| 20000 | 10.16 ± 0.05 | 10.36 ± 0.22 | 100 |
| 40000 | 10.16 ± 0.10 | 10.30 ± 0.05 | 100 |
| 60000 | 10.16 ± 0.02 | 8.17 ± 0.40 | 1.0 |

### Experimental Example 9. Analysis of Intestine-Reaching Mechanism of PEC

### 9-1. SEM-EDS Analysis

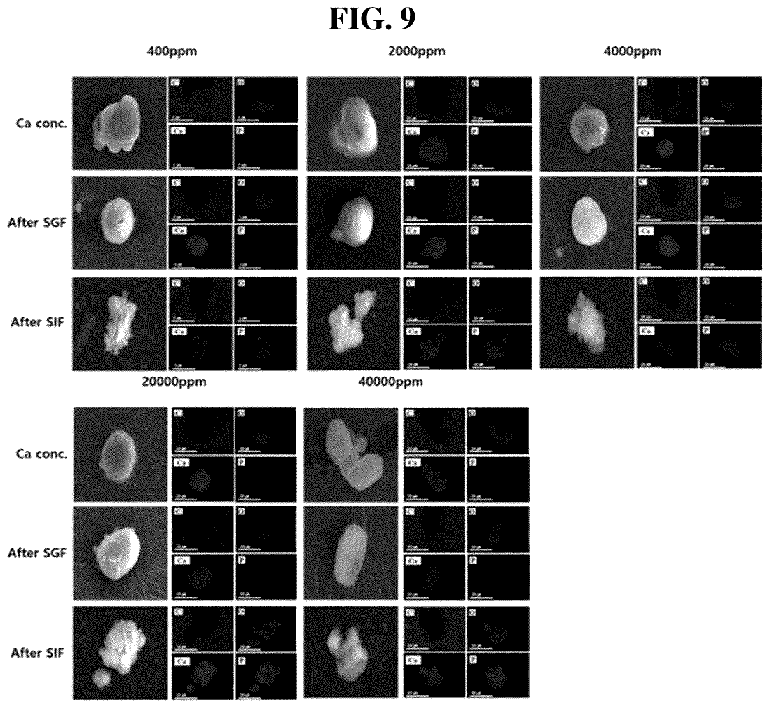

To confirm the intestine-reaching mechanism of the PEC prepared by varying the concentration of calcium solution, the acid resistance (SGF) and bile resistance (SIF) were evaluated in Experimental Example 9-1, and then the PEC was observed using SEM-EDS. PEC was prepared in the same manner as in Example 1, except that the calcium solution was by varying the concentration to 400, 2000, 4000, 20000, or 40000 ppm in Experimental Example 9-1. Next, the acid resistance and bile resistance were evaluated in the same manner as in Experimental Example 8, and the shape of the PEC was observed using SEM-EDS in the same manner as in Experimental Example 5-1. The results of Experimental Example 9-1 are illustrated in FIG. 9.

As a result of Experimental Example 9-1, it was confirmed that after the acid resistance evaluation, there was almost no change in the shape of PEC, but after the bile resistance evaluation, the shape of PEC changed to a small needle shape, which is the unique shape of hydroxyapatite (Ca₁₀(PO₄)₆(OH)₂). In addition, considering the EDS analysis results, it was confirmed that phosphorus (P) was evenly distributed on the surface of all solids obtained after the bile resistance evaluation.

### 9-2. FT-IR Analysis

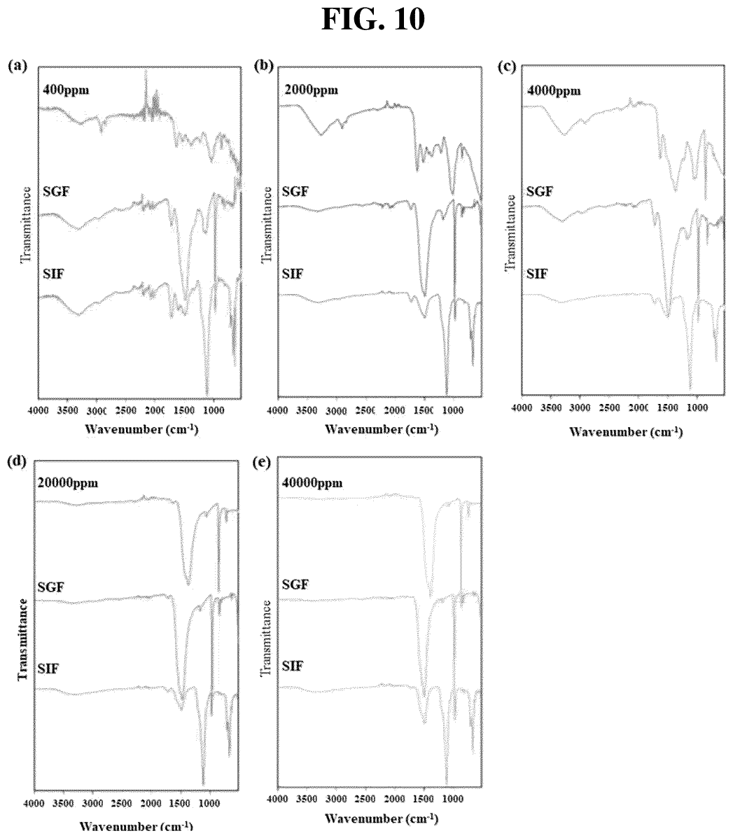

To confirm the intestine-reaching mechanism of the PEC prepared by varying the concentration of calcium solution, the PEC was analyzed by FT-IR after evaluating acid resistance (SGF) and bile resistance (SIF) in Experimental Example 9-2. PEC was prepared in the same manner as in Example 1, except that the calcium solution was prepared by varying the concentration to 400, 2000, 4000, 20000, or 40000 ppm in Experimental Example 9-2. Next, acid resistance and bile resistance were evaluated in the same manner as in Experimental Example 8, and the peak of PEC was observed by FT-IR in the same manner as in Experimental Example 3. The results of Experimental Example 9-2 are illustrated in FIG. 10.

As a result of Experimental Example 9-2, it was confirmed that the calcium carbonate of the PEC prepared by varying the concentration of the calcium solution was usually present in the form of calcite or vaterite, but it was present as calcium carbonate and hydroxyapatite after the bile resistance evaluation. More specifically, it was confirmed that the peak intensities at 1386 cm⁻¹ and 878 cm⁻¹, which are characteristics of calcite, decreased, and the peaks corresponding to the P—O bond appeared at 1048 cm⁻¹ and 565 to 600 cm⁻¹. Through this, it is considered that calcium carbonate is converted into hydroxyapatite (Ca₁₀(PO₄)₆(OH)₂) as the reaction of the following Equation 4 progresses in the bile, and probiotics are released in the process of decomposing calcium carbonate.

10CaCO₃ (s)+6H₂PO₄⁻→Ca₁₀(PO₄)₆(OH)₂ (s)+6HCO₃⁻+4CO₂+2H₂O [Equation 4]

### 9-3. XRD Analysis

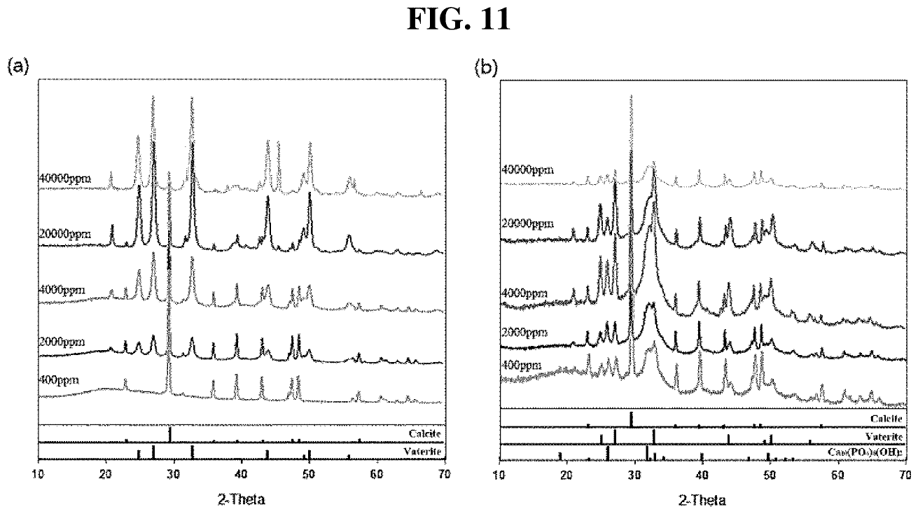

To confirm the intestine-reaching mechanism of the PEC prepared by varying the concentration of calcium solution, PEC was analyzed by XRD after acid resistance (SGF) and bile resistance (SIF) evaluation in Experimental Example 9-3. PEC was manufactured in the same manner as in Example 1, except that calcium solution was prepared by varying the concentration to 400, 2000, 4000, 20000, or 40000 ppm in Experimental Example 9-3. Next, acid resistance and bile resistance evaluations were performed in the same manner as in Experimental Example 8, and the XRD of the PEC was observed in the same manner as in Experimental Example 1-1. The results of Experimental Example 9-3 are illustrated in FIG. 11: FIG. 11A shows the XRD peak analysis results of the PEC; and FIG. 11B shows the XRD peak analysis results of the PEC after bile resistance evaluation.

As a result of Experimental Example 9-3, after the bile resistance evaluation, the peak intensity of calcium carbonate of the PEC decreased, and the peak of hydroxyapatite Ca₁₀(PO₄)₆(OH)₂ appeared.

### 9-4. XRF Analysis

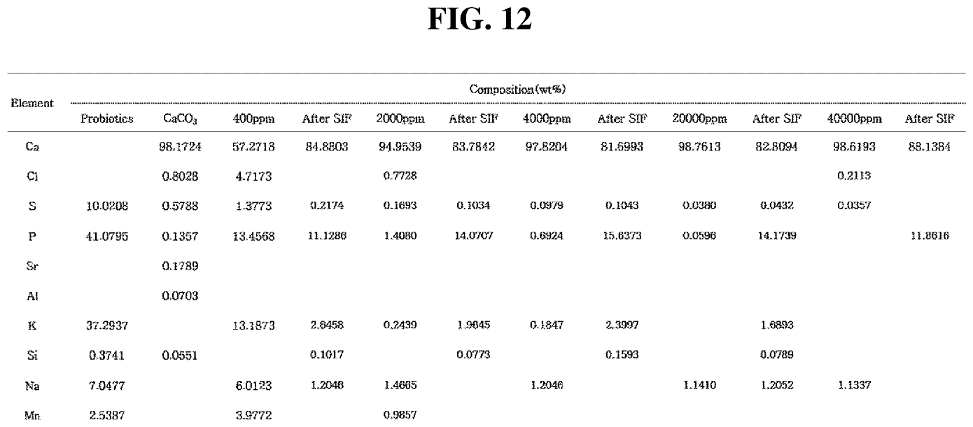

To confirm the intestine-reaching mechanism of the PEC prepared by varying the concentration of calcium solution, the acid resistance (SGF) and bile resistance (SIF) were evaluated in Experimental Example 9-4, and then the PEC was analyzed by XRF. PEC was prepared in the same manner as in Example 1, except that the calcium solution was prepared by varying the concentration to 400, 2000, 4000, 20000, or 40000 ppm in Experimental Example 9-4. Next, acid resistance and bile resistance were evaluated in the same manner as in Experimental Example 8, and the XRF of the PEC was observed in the same manner as in Experimental Example 5-2. The results of Experimental Example 9-4 are illustrated in FIG. 12.

As a result of Experimental Example 9-4, the P content of the PEC prepared in a solution having a calcium concentration of 2000 ppm or higher used for preparing the PEC was 0% to 1.4%, but the P content of the solid obtained after the SIF test significantly increased to 11.9% to 15.6%.

In summary of the results of the Experimental Examples 9-1 to 9-4, it is considered that the calcium carbonate included in the PEC of the present invention was usually present in the form of calcite or vaterite, but after the acid resistance evaluation, it was present entirely in the form of calcite, and after the bile resistance evaluation, the calcite was converted into hydroxyapatite. When the calcium carbonate of the PEC is decomposed and converted into hydroxyapatite, probiotics are released.

### Experimental Example 10. Evaluation of Storage Stability of PEC

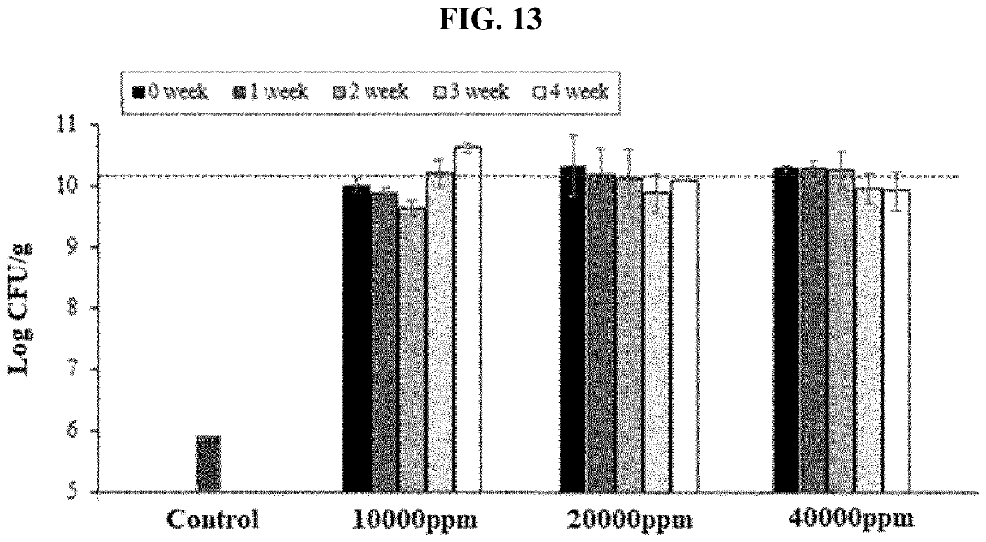

To evaluate the storage stability of PEC, Experimental Example 10 was implemented. PEC was prepared in the same manner as in Example 1, except that the calcium solution was prepared by varying the concentration to at 10,000, 20,000, or 40,000 ppm in Experimental Example 10. The prepared PEC was stored under harsh conditions (40° C., 70% humidity) for a total of four weeks, and an intestinal reach rate test was conducted every week to compare the number of viable bacteria. The intestinal reach rate test in Experimental Example 10 was conducted in the same manner as in Experimental Example 8. During the intestinal reach rate test, the probiotics were divided into a control group that was not encapsulated with calcium carbonate and an experimental group that was encapsulated with calcium carbonate. The results of Experimental Example 10 are shown as a graph in FIG. 13.

As a result of Experimental Example 10, the intestinal reach rate was 9.93 to 10.63 Log CFU/g after four weeks of storage under harsh conditions, indicating high overall storage stability. In particular, when the calcium concentration was 10,000 ppm, the intestinal reach rate was 10.63 Log CFU/g after 4 weeks, which means that all the injected probiotics survived for four weeks under the harsh conditions.

### Experimental Example 11. Verification of In Vivo Efficacy of PEC

To verify the efficacy of PEC through an in vivo animal experiment, Experimental Example 11 was implemented. More specifically, to verify the effects of PEC on the number of feces, feces weight, moisture content, and digestive tract transfer rate in a loperamide-induced constipation animal model, the effects of PEC administration on each variable were confirmed before and after inducing constipation.

### 11-1. Analysis of Effect of PEC on the Number of Feces

Loperamide is known to promote the extension of the defecation time of feces and suppress the secretion of water in the intestine, resulting in a decrease in the number of feces and the fecal weight. Therefore, to confirm the effect of PEC on the number of feces in a loperamide-induced constipation animal model, the change in the number of feces due to PEC administration was confirmed before and after inducing constipation.

The experimental animals were 6-week-old Sprague Dawley (SD) male rats in a specific pathogen-free (SPF) condition and acclimatized for seven days before use. During the acclimatization period, the diet was supplied as general solid feed, and the animals were allowed to drink filtered drinking water freely. After the acclimatization period, the experimental animals were separated using the random sampling method based on the body weight value so that the average value between groups was uniform. In Experimental Example 11-1, the experimental animals were divided into a normal group, a control group, a PEC 15 mg/kg administration group (PEC-15), a PEC 50 mg/kg administration group (PEC-50), and a PEC 150 mg/kg administration group (PEC-150), and 10 animals were used per group.

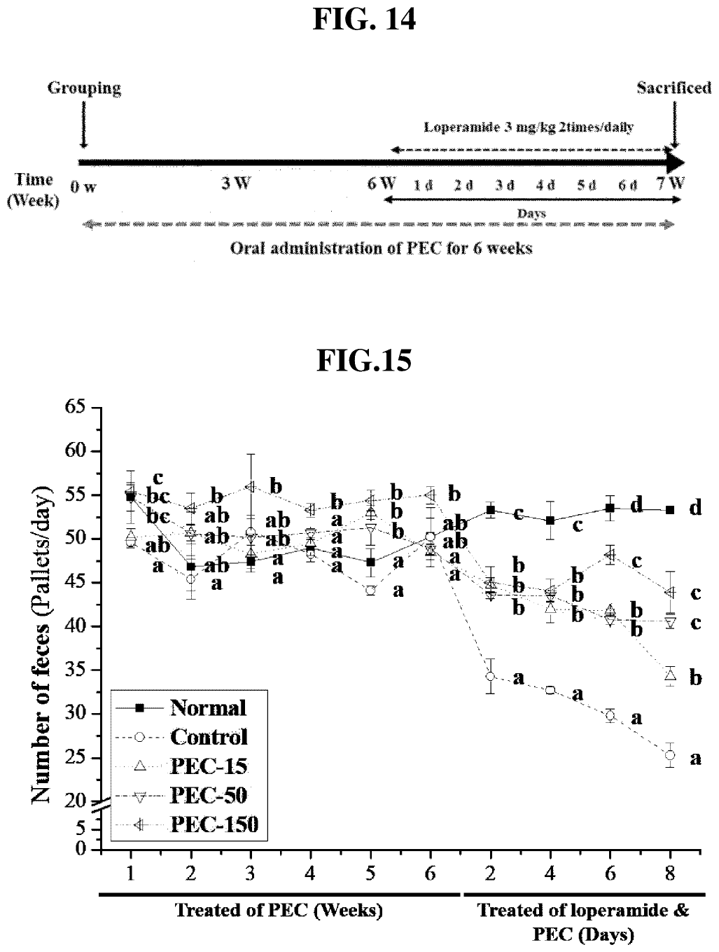

After pretreatment with PEC samples for six weeks for each experimental animal group, loperamide was administered twice a day at a concentration of 3 mg/kg for eight days, and PEC was orally administered once a day at each concentration to prepare a loperamide-induced constipation animal model. The method for creating a loperamide-induced constipation animal model is illustrated in FIG. 14.

During the six-week pretreatment period, the number of feces per day was measured once a week, and during the constipation-induced period in which loperamide was administered, the number of feces per day was measured in two-day intervals. The results of Experimental Example 11-1 are illustrated in FIG. 15.

Hereinafter, the results of Experimental Example 11-1 are described in detail.

The number of feces by experimental group according to sample pretreatment was 50.3±2.1 in the normal group, 50.2±3.4 in the control group, 48.5±0.9 in the low-concentration probiotics administration group (PEC-15), 48.9±0.6 in the medium-concentration probiotics administration group (PEC-50), and 55.0±1.0 in the high-concentration probiotics administration group (PEC-150). In other words, the PEC-150 group exhibited the largest number of feces.

On the other hand, the number of feces by experimental group on day 8, the endpoint of the experiment, was 53.3±0.2 in the normal group, 25.3±1.4 in the control group, 34.3±1.1 in the low-concentration probiotics administration group (PEC-15), 40.6±0.8 in the medium-concentration probiotics administration group (PEC-50), and 43.9±2.4 in the high-concentration probiotics administration group (PEC-150).

In other words, while the number of feces in the control group decreased over time due to constipation induction, the number of feces in the experimental group administered PEC was significantly higher than that of the control group during the constipation induction period (FIG. 15).

### 11-2. Analysis of Effect of PEC on Fecal Weight

To confirm the effect of the PEC on the fecal weight in a loperamide-induced constipation animal model, the change in the fecal weight due to PEC administration was confirmed before and after inducing constipation.

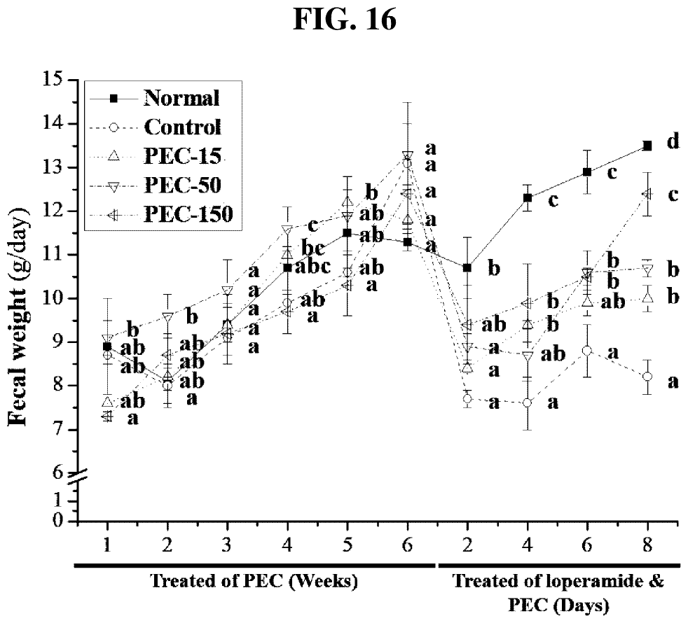

In Experimental Example 11-2, an experiment was conducted in the same manner as in Experimental Example 11-1. However, in Experimental Example 11-2, the fecal weight was measured, and all other experimental methods were the same as in Experimental Example 11-1. The results of Experimental Example 11-2 are shown as a graph in FIG. 16.

Hereinafter, the results of Experimental Example 11-2 are described in detail.

During the sample pretreatment period, the fecal weight was 11.3±0.2 g/day in the normal group, 13.1±1.4 g/day in the control group, 11.8±0.7 g/day in the low-concentration probiotics administration group (PEC-15), 13.3±0.7 g/day in the medium-concentration probiotics administration group (PEC-50), and 12.4±0.8 g/day in the high-concentration probiotics administration group (PEC-150) based on week 6. There was no significant difference in the fecal weight among the experimental groups during the sample pretreatment period.

On the other hand, the fecal weight by experimental group on day 8, the endpoint of the experiment, was 13.5±0.1 g/day in the normal group, 8.2±0.4 g/day in the control group, 10.0±0.3 g/day in the low-concentration probiotics administration group (PEC-15), 10.7±0.2 g/day in the medium-concentration probiotics administration group (PEC-50), and 12.4±0.5 g/day in the high-concentration probiotics administration group (PEC-150).

In other words, it was confirmed that the fecal weight of the control group was the lowest, and the fecal weight was significantly higher in all experimental groups administered with PEC than in the control group (FIG. 16).

### 11-3. Analysis of Effect of PEC on Fecal Moisture Content

To confirm the effect of the PEC on the fecal moisture content in a loperamide-induced constipation animal model, the change in the fecal moisture content due to PEC administration was confirmed before and after inducing constipation.

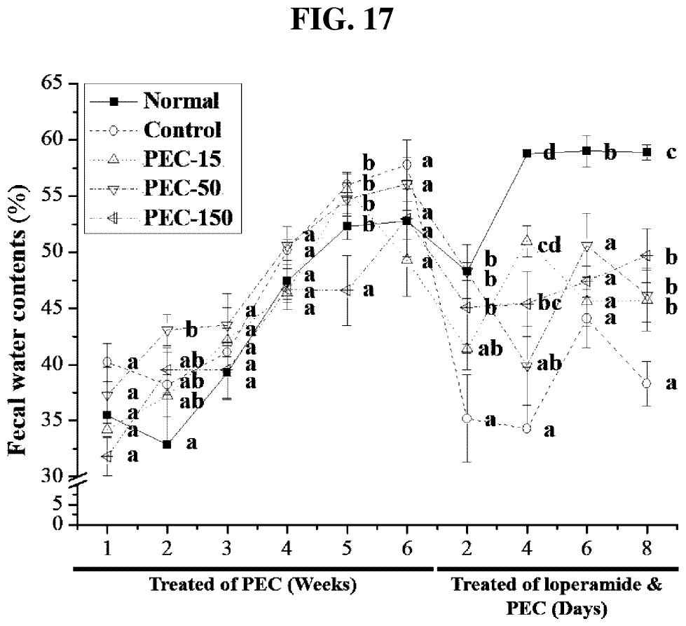

In Experimental Example 11-3, an experiment was conducted in the same manner as in Experimental Example 11-1. However, in Experimental Example 11-3, fecal moisture content was measured, and all other experimental methods were the same as in Experimental Example 11-1. The fecal moisture content was calculated as in Equation 5 by drying the collected feces in a 70° C. dryer for 24 hours, and then measuring the weight of the dried feces. The results of Experimental Example 11-3 are shown as a graph in FIG. 17.

$$Fecal moisture content ( \% ) = ( Fecal weight - dried fecal weight ) / Fecal weight \times 100 \qquad [ Equation 5 ]$$

Hereinafter, the results of Experimental Example 11-3 are described in detail.

As a result of Experimental Example 11-3, the fecal moisture content before and after inducing constipation was significantly different. On day 8, the endpoint of the experiment, the fecal moisture content of each experimental group was 58.9±0.7% in the normal group, 38.3±2.0% in the control group, 45.7±2.7% in the low-concentration probiotics administration group (PEC-15), 46.2±2.4% in the medium-concentration probiotics administration group (PEC-50), and 49.7±2.4% in the high-concentration probiotics administration group (PEC-150). In other words, the fecal moisture content of all the PEC administration groups was significantly higher than that of the control group (FIG. 17).

### 11-4. Analysis of Effect of PEC on Digestive Tract Transfer Rate

To confirm the effect of PEC on the digestive tract transfer rate in a loperamide-induced constipation animal model, the digestive tract transfer rate due to PEC administration was confirmed before and after inducing constipation. In Experimental Example 11-4, the experimental animals were prepared, PEC was administered, and a constipation-induced model was prepared in the same manner as in Experimental Example 11-1.

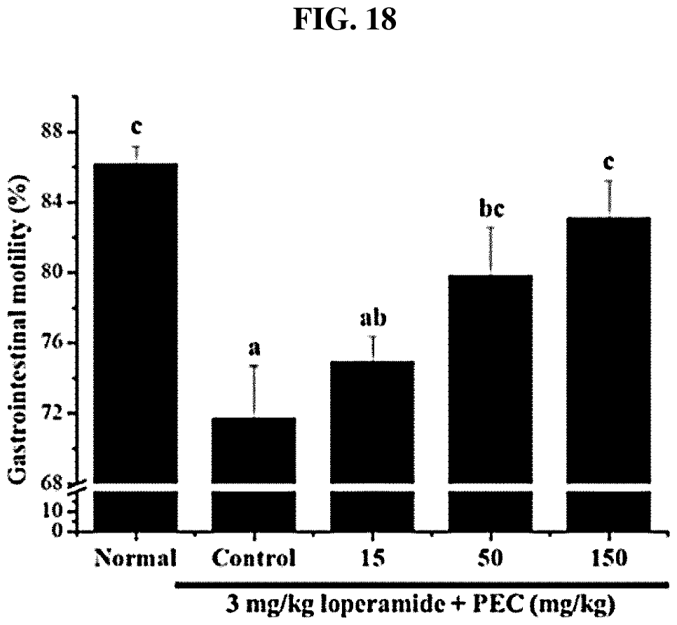

To measure the digestive tract transfer rate, the experimental animals were orally administered 10% barium sulfate after fasting for 16 hours, and an autopsy was performed after 70 minutes. The intestines were extracted during the autopsy to analyze the digestive tract transfer rate. The results of Experimental Example 11-4 are shown as a graph in FIG. 18.

As a result of Experimental Example 11-4, the digestive tract transfer rate of each experimental group was 86.16±0.97% in the normal group, 71.70±2.99% in the control group, 74.93±1.43% in the low-concentration probiotics administration group (PEC-15), 79.80±2.77% in the medium-concentration probiotics administration group (PEC-50), and 83.11±2.10% in the high-concentration probiotics administration group (PEC-150). In other words, the digestive tract transfer rate increased in the experimental group administered PEC compared to the control group, and the PEC-150 group showed a similar value to the normal group, confirming that the intestinal motility was restored to the level of the experimental group that was not administered loperamide (FIG. 18).

### Example 2. Preparation of Liquid Formulation Containing PEC

Example 2 was implemented to prepare a liquid formulation containing PEC. First, PEC was prepared in the same manner as in Example 1. Two types of liquid formulation were used in Example 2: milk (M) and milk (PM) with a pH adjusted to 7.5. To prepare the milk with a pH adjusted to 7.5, the pH was adjusted to 7.5 by adding 0.125% by weight of calcium carbonate to milk. PEC of 1% by weight (0.1 g PEC/10 mL solution) was added to each of the two liquid formulations, and the resulting mixture was stirred at a low temperature of 2 to 10° C. for 10 minutes, and stored in a refrigerator at 4° C. for four weeks.

### Experimental Example 12. Changes in pH and Intestinal Reach Rate of Liquid Formulation Containing PEC

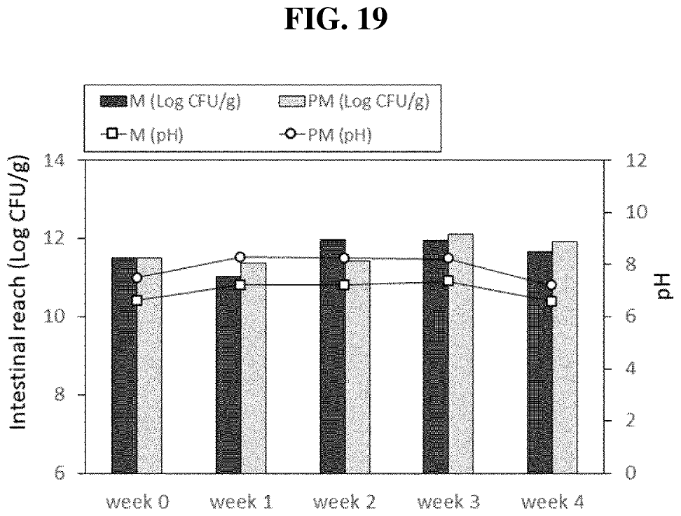

Experimental Example 12 was implemented to determine the changes in the pH and intestinal reach rate according to the storage period of the liquid formulation containing PEC prepared in above Example 2. The intestinal reach rate was measured in the same manner as in Experimental Example 8, and the results of Experimental Example 12 are shown as a graph in FIG. 19.

As a result of Experimental Example 12, most of the probiotics survived even after four weeks, and there was almost no difference in the intestinal reach rate depending on the type of liquid formulation. In addition, when the liquid formulation containing the PEC was stored at 4° C. for four weeks, 11.6 to 11.9 Log CFU/g of viable bacteria were released from the intestine, confirming that the PEC may be suitably industrialized by adding it to a liquid formulation.

From the above description, those skilled in the art will understand that the present invention can be implemented in other specific forms without changing the technical idea or essential features of the present invention. In this regard, it should be understood that the above-described embodiments are exemplary in all respects and not restrictive.

1. Probiotics encapsulated with calcium carbonate, comprising:
calcium carbonate; and
probiotics.

2. The probiotics encapsulated with calcium carbonate according to claim 1, wherein the probiotics include one or more microorganisms selected from the group consisting of Lactiplantibacillus plantarum subsp. plantarum (KCTC3108), Lacticaseibacillus casei (KCTC3110), Limosilactobacillus fermentum (KCTC3112), Bifidobacterium longum subsp. longum (KCTC3128), Lactobacillus gasseri (KCTC3144), Lactobacillus acidophilus (KCTC3164), Lactobacillus paragasseri (KCTC3172), Lacticaseibacillus rhamnosus (KCTC3237), Bifidobacterium breve (KCTC3419), Limosilactobacillus reuteri (KCTC3594), Lactobacillus delbrueckii subsp. bulgaricus (KCTC3635), Lactococcus lactis subsp. lactis (KCTC3769), Enterococcus faecalis (KCTC5191), Bifidobacterium animalis subsp. lactis (KCTC5854), Enterococcus faecium (KCTC13225), and Lactobacillus helveticus (KCTC15060).

3. The probiotics encapsulated with calcium carbonate according to claim 1, wherein the probiotics encapsulated with calcium carbonate have a particle size of 0.9 to 9.2 μm.

4. The probiotics encapsulated with calcium carbonate according to claim 1, wherein the probiotics encapsulated with calcium carbonate have a calcium carbonate content of 17% to 98%.

5. The probiotics encapsulated with calcium carbonate according to claim 1, wherein the probiotics encapsulated with calcium carbonate have an intestine-reaching cell count of 8 to 11 Log CFU/g.

6. The probiotics encapsulated with calcium carbonate according to claim 1, wherein the calcium carbonate reacts with bile and is converted into hydroxyapatite.

7. The probiotics encapsulated with calcium carbonate according to claim 1, wherein the probiotics encapsulated with calcium carbonate have an encapsulation yield of 96% to 100%, and the encapsulation yield is calculated by Equation 1 below:
$$E ( \% ) = [ ( {N}_{0} - {N}_{1} ) \div {N}_{0} ] \times 100 \qquad [ Equation 1 ]$$
(In the calculation formula, E(%) is the encapsulation yield, N₀ is the number of cells used in the probiotics encapsulation process (CFU/g), and N₁ is the number of cells remaining in the solution after encapsulation (CFU/g), that is, the number of cells that have not settled inside capsules (CFU/g).)

8. Probiotic powder prepared by freeze-drying the probiotics encapsulated with calcium carbonate according to claim 1.

9. A liquid probiotic formulation prepared by adding the probiotics encapsulated with calcium carbonate according to claim 1 to a liquid formulation.

10. A method for preparing probiotics encapsulated with calcium carbonate, comprising:
culturing probiotics; and
mixing a calcium source, a carbonate source, and the cultured probiotics and allowing them to react to produce probiotics encapsulated with calcium carbonate.

11. The method for preparing probiotics encapsulated with calcium carbonate according to claim 10, further comprising:
freeze-drying and powdering the probiotics encapsulated with calcium carbonate.

12. The method for preparing probiotics encapsulated with calcium carbonate according to claim 10, wherein the calcium source is one or more calcium sources selected from the group consisting of CaCl₂, Ca(NO₃)₂, CaO, and organic acid calcium.

13. The method for preparing probiotics encapsulated with calcium carbonate according to claim 10, wherein the carbonate source is one or more carbonate sources selected from the group consisting of Na₂CO₃, K₂CO₃, (NH₄)₂CO₃, and CO₂ (g).

14. The method for preparing probiotics encapsulated with calcium carbonate according to claim 10, wherein the probiotics include one or more microorganisms selected from the group consisting of Lactiplantibacillus plantarum subsp. plantarum (KCTC3108), Lacticaseibacillus casei (KCTC3110), Limosilactobacillus fermentum (KCTC3112), Bifidobacterium longum subsp. longum (KCTC3128), Lactobacillus gasseri (KCTC3144), Lactobacillus acidophilus (KCTC3164), Lactobacillus paragasseri (KCTC3172), Lacticaseibacillus rhamnosus (KCTC3237), Bifidobacterium breve (KCTC3419), Limosilactobacillus reuteri (KCTC3594), Lactobacillus delbrueckii subsp. bulgaricus (KCTC3635), Lactococcus lactis subsp. lactis (KCTC3769), Enterococcus faecalis (KCTC5191), Bifidobacterium animalis subsp. lactis (KCTC5854), Enterococcus faecium (KCTC13225), and Lactobacillus helveticus (KCTC15060).

15. The method for preparing probiotics encapsulated with calcium carbonate according to claim 10, wherein the calcium source has a concentration of 50 to 60000 ppm.

16. The method for preparing probiotics encapsulated with calcium carbonate according to claim 10, wherein the carbonate source has a concentration of 50 to 60000 ppm.
